(19) United States
(12) Patent Application Publication
(10) Pub. No.: US 2003/0119719 A1
(43) Pub. Date: Jun. 26, 2003

(54) Use of endothelin conjugates in therapy, new endothelin conjugates, agents that contain the latter, and process for their production

(76) Inventors: Ludger Dinkelborg, Berlin (DE); Ulrich Speck, Berlin (DE); Christoph-Stephan Hilger, Berlin (DE); Friedhelm Blume, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/988,008

(22) Filed: Nov. 16, 2001

Related U.S. Application Data
(63) Continuation of application No. 09/319,414, filed on Nov. 26, 1999, now abandoned.

(30) Foreign Application Priority Data
Oct. 4, 1996 (DE) 196 52 374.5
Nov. 24, 1997 (EP) PCT/EP97/06518

(51) Int. Cl. A61K 51/00; A61K 39/395; A61K 38/17; A61K 38/16

(52) U.S. Cl. 514/2; 514/8; 514/9; 424/1.49; 424/178.1

(57) ABSTRACT

This invention relates to the use of conjugates that consist of endothelins and active groups for therapy of vascular diseases as well as new endothelin conjugates, agents that contain these compounds and process for their production.

The invention relates to the subject that is characterized in the claims, i.e., the use of conjugates from radicals that bind to endothelin receptors and active groups for therapy of diseases.

The invention relates especially to the use of conjugates that consist of endothelin derivatives, partial sequences of endothelins, endothelin analogs, or endothelin antagonists and an active group for the therapy of vascular diseases.

Another aspect of the invention relates to new endothelin conjugates, agents that contain these compounds, and a process for their production.

Cardiovascular diseases are one of the most common diseases in industrialized countries. They represent one of the most frequent causes of death. In most cases, cardiovascular diseases are caused by arteriosclerosis. This is an inflammatory, fibroproliferative disease, which is responsible for 50% of all deaths in the USA, Europe, and Japan (Ross 1993, Nature 362: 801-809). In its peripheral manifestation, it threatens the upkeep of the extremities; in its coronary manifestation, there is the risk of fatal myocardial infarction; and in a supra-aortic attack, there is the threat of stroke.

At this time, the treatment of arteriosclerosis is done in different ways. Thus, in addition to conservative measures (e.g., lowering of the cholesterol level in the blood) and bypass operations, mechanical dilatation (angioplasty), as well as the intravascular removal of atheromatous tissue (atherectomy) of stenotic segments in peripheral arteries and the coronaries, have become established as alternatives in regular clinical practice.

As indicated below, however, the above-mentioned methods are associated with a wide variety of drawbacks.

Thus, the value of the mechanical rechanneling process is acutely impaired by vascular occlusions as a result of vascular lacerations and dissections, as well as acute thromboses (Sigwart et al. 1987, N. Engl. J. Med. 316: 701-706). Long-term success is jeopardized by the reoccurrence of constrictions (restenosis). Thus, the CAVEAT study showed that of 1012 patients, the restenosis rate was 50% in the case of coronary atherectomy six months after intervention and even 57% in the case of coronary angioplasty (Topol et al. 1993, N. Engl. J. Med. 329: 221-227). In addition, in this study sudden vascular occlusions occurred in 7% of the atherectomy patients and in 3% of the angioplasty patients. Nicolini and Pepine (1992, Endovascular Surgery 72: 919-940) report a restenosis rate of between 35 and 40% and an acute closure rate of 4% after angioplastic intervention.

To counteract these complications, different techniques were developed. This includes the implantation of metallic endoprostheses (stents) (Sigwart et al. 1987, N. Engl. J. Med. 316: 701-706; Strecker et al., 1990, Radiology 175: 97-102). Stent implantation in large-caliber arteries, e.g., in the case of occlusions in the axis of the pelvis, has already become a mode of treatment that is to be used primarily. The use of stents in femoral arteries, however, with a primary openness rate of 49% and a reclusion frequency of 43%, has provided disappointing results (Sapoval et al., 1992, Radiology 184:833-839). Similar unsatisfactory results were achieved with previously available stents in the coronary arteries (Kavas et al. 1992, J. Am. Coll. Cardiol 20: 467-474).

Up until now, all previous pharmacological and mechanical interventions have been unable to prevent restenosis (Muller et al. 1992, J. Am. Coll. Cardiol. 19: 418-432, Popma et al. 1991, Circulation 84: 14226-1436).

The reason for the restenoses that occur frequently after mechanical interventions is assumed to be that the interventions induce a proliferation and migration of unstriped muscle cells in the vessel wall. The latter result in a neointimal hyperplasia and the observed restenoses in the treated vessel sections (Cascells 1992, Circulation 86, 723-729, Hanke et al. 1990, Circ. Res. 67, 651-659, Ross 1986, Nature 362, 801-809, Ross 1993, Nature 362, 801-809).

An alternative process for treating arteriosclerotic diseases involves ionizing radiation. It is known that ionizing radiation inhibits the proliferation of cells. A considerable number of neoplastic and non-neoplastic diseases have already been treated in this way (Fletcher, Textbook of Radiotherapy, Philadelphia, Pa.: Lea and Febiger, 1980, Hall, Radiobiology for the Radiologist, Philadelphia, Pa.: Lippincott, 1988).

The use of ionizing radiation of external origin on restenoses is, however, associated with the drawback that, when administered, the radiation dose at the desired site is small and, moreover, surrounding (healthy) tissue is also exposed to the radiation in an undesirable way. Thus, up until now, various studies have not provided very promising results (Gellmann et al. 1991, Circulation 84 Suppl. II: 46A-59A, Schwartz et al. 1992, J. Am. Coll. Cardiol. 19:1106-1113).

These drawbacks, which arise when external radiation sources are used, can be overcome if gamma radiation is transported directly, e.g., via a catheter, to the vessel areas with restenosis. With this form of administration, a high radiation dose of 20 Gy/h is transported to the restenosis foci with iridium-192. Some papers report almost complete prevention of restenosis after this intervention (Wiedermann et al. 1994, Am. J. Physiol. 267:H125-H132, Böttcher et al. 1994, Int. J. Radiation Oncology Biol. Phys. 29:183-186, Wiedermann et al. 1994, J. Am. Coll. Cardiol. 23: 1491-1498, Liermann et al. 1994, Cardiovasc. Intervent. Radiol. 17: 12-16). A drawback of this method is, however, that the radiation dose of 20 Gy/h that is administered in this case is very high. Since the lesions are dispersed irregularly on the vessel wall, uniform administration of a defined dose is not possible with the aid of this technique. In addition, treatment of large-caliber vessels is not possible since the dose that can be administered is not sufficient because of the drop in the dose from the iridium source.

Another possibility for inhibiting restenosis is the implantation of P-32-coated stents (Fischell et al. Stents III, Entwicklung, Indikationen und Zukunft, Konstanz [Development, Indications and the Future: Constancy]: Kollath and Liermann, 1995). In this paper, an activity of 0.2 kBq of P-32 per centimeter of stent length (corresponds to a radiation dose of 0.25 Gy) was sufficient to ensure maximum inhibition of the unstriped muscle cells in vitro. It was thus possible to show that not only γ- but also β-emitters prevent the proliferation of unstriped muscle cells. The advantage of this method is that the dose of radiation administered is considerably lower than with all the types of intervention mentioned to date. At this low dose, the endothelial cells that line the vascular bed are not damaged (Fischell et al. Stents III, Entwicklung, Indikationen und Zukunft, Konstanz: Kollath and Liermann, 1995). This form of intervention is possible, however, only once, namely during the positioning of the stent. Moreover, it is limited to only those interventions in which stents are used. The restenoses that occur in the far more common interventions such as atherectomies and angioplasties cannot be treated by this method. Because of the small range of action of the β-radiation, it is not possible to administer a uniform dose of energy to the entire lesion. Finally, up until now, it has not been possible to resolve the problem of coating stents in a stable manner with isotopes, such as, e.g., P-32.

In addition to radiation therapy, a number of other therapeutic strategies are also used for inhibiting neointimal hyperplasias (restenoses). These include standard medications for restenosis suppression such-as antithrombotic agents, platelet aggregation inhibitors, calcium antagonists, anti-inflammatory and anti-proliferative substances, but also gene-therapy approaches. In this connection, the inhibition of growth stimulators is possible with, e.g., antisense oligonucleotides or the enhancement of inhibiting factors by expression-vector-plasmids and virus-mediated gene integration. Also, aptamer oligonucleotides can be used to inhibit a wide variety of receptor-mediated processes, which play a decisive role in restenosis.

Over the years a great deal of energy and effort has gone into studying substances that were administered under strictly controlled conditions as long-term therapy since researchers hoped to find a way to reduce the restenosis rate (Herrmann et al., 1993, Drugs 46: 18-52).

More than 50 controlled studies with different substance groups were carried out, without yielding definite proof that the investigated substances could significantly reduce the restenosis rate. This applies also for topical application, with which the substances are brought via special balloon catheter to the site of action that is desired in each case. It has been shown, however, that the substances are washed out from the vessel wall too quickly to be therapeutically effective. Moreover, these pressure-mediated liquid injections induce additional vessel wall alterations that promote restenosis.

Other therapeutic approaches take advantage of the fact that increased cell proliferation is observed in the case of arteriosclerotic diseases. Thus, recent studies have demonstrated elevated tyrosine kinase activity in cell proliferation processes (Bishop 1987, Science 335, 305-314, Ross 1986, N. Engl. J. Med. 314, 488-500, Ross 1993, Nature 362, 801-809). By using specific inhibitors of protein tyrosine kinases (PTK), cell proliferation processes should be slowed.

The inhibition of PTK activity is, however, not free of side-effects since PTKs are also responsible for normal proliferation and metabolic processes (e.g., insulin receptor or NGF receptor) (Levitzki 1992, FASEB 6, 3275-3282).

The inadequate dwell time of the PTK blockers as well as their insufficient selectivity represent another unresolved problem. In addition, all PTK blockers must be able to pass through the cell membrane in order to be effective.

In addition to PTK blockers, cytostatic agents, such as, e.g., cis-diaminedichloroplatinum (cis-platinum), are also used to treat neoplastic diseases (Rozencweig et al., 1977. Ann. Intern. Med., 86, 803-812). Although cis-platinum has proven to be a very effective therapeutic agent for the above-mentioned purpose, it cannot be widely used since the therapeutic window of this substance is greatly limited by the various, sometimes drastic systemic side-effects. Primarily the nephrotoxic effect of renally eliminated cis-platinum is responsible for the limited clinical use of this substance (Dentino et al. 1987, Cancer 41, 1274-1281, Groth et al. 1986, Cancer Chemother. Pharmacol. 17, 191-196).

It was therefore the object of this invention to find compounds that are suitable for therapeutic treatment of cardiovascular diseases, especially for the treatment of vascular diseases, such as, e.g., arteriosclerosis, and that overcome the drawbacks of the compounds of the prior art.

This object is achieved by this invention.

It has been found that conjugates of endothelins and at least one active group are extremely well suited for therapy, especially for therapy for vascular diseases.

The term endothelin conjugate is also understood to encompass conjugates of endothelin derivatives, partial sequences of endothelins, endothelin analogs, or endothelin antagonists.

The invention thus relates to the use of endothelin conjugates for therapeutic treatment of vascular diseases.

Another aspect of the invention relates to new conjugates of endothelins, endothelin derivatives, partial sequences of endothelins, endothelin analogs, or endothelin antagonists and at least one active group, a process for their production, agents that contain these conjugates, and their use in diagnosis and therapy.

It has been found that conjugates of endothelins, endothelin derivatives, partial sequences of endothelins, endothelin analogs, or endothelin antagonists and an active group are concentrated in cells and tissues in which endothelin receptors are more strongly expressed. These receptors are found especially in arteriosclerotic deposits (plaque). Surprisingly enough, despite being coupled to an active group, endothelins retain their high specificity relative to these receptors, so that even at a low dosage, a therapeutically effective concentration of the active group can be achieved at the target site. The retention time of the conjugates is also long enough to accomplish the desired therapeutic effect. At this dosage, the concentration in other tissues does not reach any toxic range, particularly because the conjugates that contain active groups that do not bind to the unstriped muscle cells are quickly eliminated from the body and thus the stress on the patient that is caused by an unbonded conjugate is minimal. The observed systemic side-effects are therefore slight.

Surprisingly enough, moreover, some of the conjugates according to the invention are taken up in the cell after binding to the receptors as a substance-receptor complex. Thus, it is possible not only to transport the active groups specifically to the foci of disease, but also to deposit them intracellularly. Mainly in the case of such active groups, which are less readily compatible and in particular exert their actions intracellularly, this is of decisive advantage for therapy.

As endothelins, endothelin derivatives, partial sequences of endothelins, endothelin analogs, or endothelin antagonists, the following structures can be mentioned by way of example:

```
                    \overline{| |}
Cys-Ser-Cys-Ser-Ser-Leu-Met-Asp-Lys-Glu-Cys-Val-Tyr-
|
\overline{ |}
          |
Phe-Cys-His-Leu-Asp-Ile-Ile-Trp.
                    \overline{| |}
Cys-Ser-Cys-Set-Ser-Trp-Leu-Asp-Lys-Gtu-Cys-Val-Tyr
  |
  \overline{ |}
Phe-Cys-His-Leu-Asp-Ile-Ile-Trp.
                    \overline{| |}
Cys-Thr-Cys-Phe-thr-Tyr-Lys-Asp-Lys-Glu-Cys-Val-Tyr-
  |
  \overline{ |}
Tyr-Cys-His-Leu-Asp-Ile-Ile-Trp.
                    \overline{| |}
Cys-Ser-Cys-Ser-Ser-Trp-Leu-Asp-Lys-Glu-Cys-VaI-Tyr-
  |
  \overline{ |}
Phe-Cys-His-Leu-Asp-lle-lle-Trp.
                    \overline{| |}
Cys-Thr-Cys-Phe-Thr-Tyr-Lys-Asp-Lys-Glu-Cys-Val-Tyr
  |
  \overline{ |}
Tyr-Cys-His-Leu-Asp-Ile-Ile-Trp.
Cys-Ser-Ala-Ser-Ser-Leu-Met-Asp-Lys-Glu-Ala-Val-Tyr-
  |
  \overline{ |}
Phe-Cys-His-Leu-Asp-Ile-lle-Trp.
                    \overline{| |}
Cys-Ser-Cys-Asn-Ser-Trp-Leu-Asp-Lys-Glu-Cys-Val-Tyr-
  |
  \overline{ |}
Phe-Cys-His-Leu-Asp-Ile-Ile-Trp,
                    \overline{| |}
Cys-Ser-Cys-Lys-Asp-Met-Thr-Asr-Lys-Glu-Cys-Leu-Asn-
  |
  \overline{ |}
Phe-Cys-His-Gln-Asp-Val-Ile-Trp,
                    \overline{| |}
Ala-Ser-Cys-Ser-Ser-Leu-Met-Asp-Lys-Glu-Cys-VaI-Tyr-
Phe-AIa-His-Leu-Asp-Ile-Ile-Trp.
Ala-Ser-Ala-Ser-Ser-Leu-Asp-Lys-Glu-Ala-Val-Tyr-Phe-Ala-His-Leu-Asp-Ile-Ile
Trp.
Cys-Ser-Cys-Ser-Ser-Trp-Leu-Asp-Lys-Glu-Ala-Val-Tyr-Phe-Ala-His-Leu-Asp-Ile-Ile-
Trp.
  \overline{| |}
Cys-Val-Tyr-Phe-Cys-His-Leu-Asp-Ile-Ile-Trp.
N-Acetyl-Leu-Met-Asp-Lys-Glu-Ala-Val-Tyr-Phe-Ala-His-Leu-Asp-Ile-Ile-Trp.
His-Leu-Asp-Ile-Ile-Trp.
(DTrp)-Leu-Asp-Ile-Ile-Trp.
Cyclo-(DTrp-DAsp-Pro-DVal-Leu),
Cyclo-(DGlu-Ala-alloDIle-Leu-DTrp).
Cyclo-(D-Trp-D-Asp-Pro-α-(2-thienyl)-D-Gly-Leu).
H-Gly-Asn-Trp-His-Gly-Thr-Ala-Pro-Asp-Trp-Val-Tyr-Phe-Ala-His-Leu-Asp-Ile-Ile-
Trp-OH.
                    \overline{| |}
Cys-Thr-Cys-Asn-Asp-Met-Tyr-Ala-Glu-Glu-Cys-Leu-Asn-
|
\overline{ |}
Phe-Cys-His-Glu-Asp-Val-Ile-Trp.
Glu-Ala-Val-Tyr-Phe-AIa-His-Leu-Asp-Ile-Ile-Trp.
Ac-Leu-Met-Asp-Lys-Glu-Ala-Val-Tyr-Phe-Ala-His-Leu-Asp-Ile-Ile-Trp.
Suc-Asp-Glu-Glu-Ala-Val-Thr-Phe-Ala-His-Leu-Asp-Ile-Ile-Trp.
  \overline{| |}
Cys-Val-Tyr-Phe-Cys-His-Asp-Leu-Ile-Ile-Trp.
                    \overline{| |}
Cys-Ser-Cys-Ser-Ser-Leu-Met-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Ser-Ser-Leu-Met-Asp-Lys-Glu-Cys-VaI-Tyr-
|
\overline{ |}
Phe-Cys-His-Leu-Thr-γ-methyl-Leu-Ile-Trp.
Leu-Asp-Ile-Ile-Trp.
Ac-His-Leu-Asp-Ile-Ile-Trp.
Ac-D-His-Leu-Asp-Ile-Ile-Trp.
Ile-Ile-Trp.
Asp-Gly-Gly-Cys-Gly-Cys-(D-Trp)-Leu-Asp-Ile-Ile-Trp.
Asp-Gly-Gly-Cys-Gly-Cys-Phe-(D-Trp)-Leu-Asp-Ile-Ile-Trp.
```

Ac-D-Bhg-Leu-Asp-Ile-Ile-Trp, in which Bhg stands for a 10,11-dihydro-5H-dibenzo-[a,d]-cyclohepteneglycine radical,

Ac-D-Bip-Leu-Asp-Ile-Ile-Trp, in which Bip stands for a 4,4′-biphenylalanine radical or a 4-t-butyl-N-[6-(2-hydroxy-ethoxy)-5-(3-methoxy-phenoxy)-4-pyrimidinyl-benzenesulfonamide radical,

a 4-t-butyl-N-[6-(1′,2′-dihydroxy-propyloxy)-5′-(2-methoxy-phenoxy)-2-methoxy-4-pyrimidinyl-benzenesulfonamide radical,

a 4-t-butyl-N-[6′-(2′-hydroxy-ethoxy)-5-(2-ethoxy-phenoxy)-2,2′-bipyrimidin-4-yl-benzenylsulfonamide radical,

a 27-O-caffeoylmyricerone radical or

a 2(R)-[2-(R)-[2(S)-[[1-(hexahydro-1H-azepinyl)]carbonyl]amino-4-methylpentanoyl]amino-3-[1-methyl-1H-indonyl)]propinonyl]amino-3-(2-pyridyl)propionic acid radical.

As active groups, antibodies, antibody fragments, peptides, carbohydrates, oligonucleotides, hormones, or chemotherapy agents are suitable. The active groups, however, can also be radioactive metal isotopes and their metal complexes, as well as radioactive isotopes of various non-metals, whereby the latter are bonded to the endothelin either directly or via a suitable radical.

According to the invention, conjugates with one or more, preferably 1 to 10, active groups or active ingredient molecules can be used.

As chemotherapy agents, there can be mentioned by way of example vinblastine, doxorubicin, bleomycin, methotrexate, 5-fluorouracil, 6-thioguanine, cytarabine, cyclophosphamide and cis-platinum, as well as other conventional chemotherapy agents (see, e.g., Cancer: Principles and Practice of Oncology, 2nd Ed., V. T. De Vita, Jr.; S. Hellman; S. A. Rosenberg, J. B. Lippincot Co., Philadelphia, Pa., 1985, Chapter 14). Among the above-mentioned, cis-platinum is preferred.

In addition, pharmaceutical agents that are used in experimental studies are suitable as active groups, such as, e.g., mercaptopurine, N-methyl-formamide, 2-amino-1,3,4-thiadiazole, melphalan, hexamethylmelanine, dichloromethotrexate, mitoguazone, sumarin, bromodeoxyuridine, iododeoxyuridine, semustine, 1-(2-chloroethyl)-3-(2,6-dioxo-3-piperidyl)-1-nitrosourea, N,N′-hexamethylene-bis-acetamide, azacytidine, dibromodulcitol, erwinia-asparaginase, ifosfamide, 2-mercaptoethanesulfonate, teniposide, taxol, 3-deazauridine, soluble Baker's folic acid antagonist, homoharringtonine, cyclocytidine, acivicin, ICRF-187, spiromustine, levamisole, chlorozotocin, aziridinylbenzoquinone, spirogermanium, aclarubicin, pentostatin, PALA, carboplatinum, amsacrine, caracemide, iproplatin, misonidazole, dihydro-5-azacytidine, 4′-deoxy-doxorubicin, menogaril, triciribine phosphate, fazarabine, tiazofurin, teroxirone, ethiofos, N-(2-hydroxyethyl)-2-nitro-1H-imidazole-1-acetamide, mitoxantrone, acodazole, amonafide, fludarabine phosphate, pibenzimol, didemnin B, merbarone, dihydrolene perone, flavone-8-acetic acid, oxantrazole, ipomeanol, trimetrexate, deoxyspergualin, echinomycin and dideoxycytidine (cf., NCI Investigational Drugs, Pharmaceutical Data 1987, NIH Publication No. 88-2141, Revised November 1987).

In addition, antithrombotic agents are suitable as active groups, such as, e.g., heparin, hirudin, low molecular weight heparin or marcumar; growth factor inhibitors, such as, e.g., anti-PDGF, [e.g., triazolopyrimidine (Trapidil^{(R)})]; platelet aggregation inhibitors, such as, e.g., RGD-peptides, which bind to GP IIb/IIIa receptors, acetylsalicylic acid (Aspirin^{(R)}), dipyridamole, thrombin, clotting cascade inhibitors, such as, e.g., factor VIIa or Xa inhibitors; anti-inflammatory agents, such as, e.g., corticoids or nonsteroidal anti-inflammatory agents; Ca antagonists such as, e.g., verapamil, nifedipine or diltiazem; lipid-lowering agents, such as, e.g., simvastatin or probucol; anti-proliferative agents such as, e.g., colchicine, angiopeptin, estradiol or ACE inhibitors (e.g., Ramipril^{(R)}); antisense oligonucleotides; aptamer oligonucleotides; PTK blockers such as, e.g., quercetin, genistein, erbstatin, lavendustin A, herbimycin A or aeroplysinin-1 or synthetic PTK blockers, such as, e.g., tyrphostins, S-aryl-benylidene malononitrile compounds, or benzylidene malononitrile (BMN) compounds.

As active groups, groups that contain radionuclides are especially suitable. Radionuclides that can be used according to the invention include alpha-, beta- and/or gamma-radiators, positron radiators, Auger electron radiators, and fluorescence radiators, whereby beta- or alpha-radiators are preferable for therapeutic purposes.

Corresponding radionuclides are known to one skilled in the art. By way of example, there can be mentioned the radionuclides of the elements Ag, As, At, Au, Ba, Bi, Br, C, Co, Cr, Cu, F, Fe, Ga, Gd, Hg, Ho, I, In, Ir, Lu, Mn, N, O, P, Pb, Pd, Pm, Re, Rh, Ru, Sb, Sc, Se, Sm, Sn, Tb, Tc or Y.

The binding of the radionuclide to the endothelin radical is carried out either directly or—especially in the case of metallic radionuclides, such as, e.g., a nuclide of the elements Ag, As, Au, Bi, Cu, Ga, Gd, Hg, Ho, In, Ir, Lu, Pb, Pd, Pm, Pr, Re, Rh, Ru, Sb, Sc, Se, Sm, Sn, Tb, Tc or Y—with a corresponding complexing agent, which is coupled to the endothelin.

Suitable endothelin conjugates with metal complexes are described by, i.a., Dinkelborg et al. [J. N. M. 36 (1995) 102], as well as in DE-43 01 871 and DE-44 25 778. The conjugates are used in the diagnosis of diseases, especially in the diagnosis of arteriosclerosis.

Since the drop in the dose is very steep in the case of β-emitters, isotopes that emit both β- and γ-radiation (such as, e.g., rhenium isotopes) are especially preferred.

Conjugates with radionuclides that emit γ-radiation are also suitable since their dosage can be easily monitored by radiodiagnostic methods.

Another aspect of the invention relates to new endothelin conjugates of formula II

E-W¹ₙ (II)

in which E stands for a radical that binds endothelin receptors and is derived from endothelins, endothelin analogs, endothelin derivatives, endothelin partial sequences, and endothelin antagonists, and W¹ stands for an active group that contains a radionuclide of the elements At, Ba, Br, C, F, N, O or P or that is derived from a chemotherapy agent, an antibody, antibody fragment, peptide, carbohydrate, oligonucleotide, PTK blocker, antithrombotic agent, growth factor inhibitor, pharmaceutical agent, platelet aggregation inhibitor, anti-inflammatory agent, Ca-antagonist, lipid-lowering agent, or an antiproliferative agent, and n stands for numbers 1 to 100, preferably 1 to 10.

As radicals that bind endothelin receptors, the following structures preferably can be mentioned:

```
                    \overline{| |}
Cys-Ser-Cys-Ser-Ser-Leu-Met-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Ser-Ser-Trp-Leu-Asp-Lys-Glu-Cys-Val-Tyr-
|
\overline{ |}
Phe-Cys-His-Leu-Asp-Ile-Ile-Trp.
                    \overline{| |}
Cys-Thr-Cys-Phe-Thr-Tyr-Lys-Asp-Lys-Glu-Cys-Val-Tyr-
|
\overline{ |}
Tyr-Cys-His-Leu-Asp-Ile-Ile-Trp.
                    \overline{| |}
Cys-Ser-Cys-Ser-Ser-Trp-Leu-Asp-Lys-Glu-Cys-Val-Tyr-
|
\overline{ |}
Phe-Cys-his-Leu-Asp-Ile-Ile-Trp.
                    \overline{| |}
Cys-Thr-Cys-Phe-Thr-Tyr-Lys-Asp-Lys-Glu-Cys-Val-Tyr-
|
\overline{ |}
Tyr-Cys-His-Leu-Asp-Ile-Ile-Trp.
Cys-Ser-Ala-Ser-Ser-Leu-Met-Asp-Lys-Glu-Ala-Val-Tyr-
|
\overline{ |}
Phe-Cys-His-Leu-Asp-Ile-Ile-Trp.
                    \overline{| |}
Cys-Ser-Cys-Asn-Ser-Ttp-Leu-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Lys-Asp-Met-Thr-Asp-Lys-Glu-Cys-Leu-Asn-
|
\overline{ |}
Phe-Cys-His-Gln-Asp-Val-Ile-Trp.
                    \overline{| |}
Ala-Ser-Cys-Ser-Ser-Leu-Met-Asp-Lys-Glu-Cys-Val-Tyr-
Phe-Ala-His-Leu-Asp-Ile-Ile-Trp.
Ala-Ser-Ala-Ser-Ser-Leu-Met-Asp-Lys-Glu-Ala-Val-Tyr-Phe-Ala-His-Leu-Asp-Ile-Ile-
Trp.
Cys-Ser-Cys-Ser-Ser-Trp-Leu-Asp-Lys-Glu-Ala-Val-Tyr-Phe-Ala-His-Leu-Asp-Ile-Ile-
Trp.
\overline{| |}
Cys-Val-Tyr-Phe-Cys-His-Leu-Asp-Ile-Ile-Trp.
N-Acetyl-Leu-Met-Asp-Lys-Glu-Ala-Val-Tyr-Phe-Ala-His-Leu-Asp-Ile-Ile-Trp.
His-Leu-Asp-Ile-Ile-Trp.
(D-trp)-Leu-Asp-Ile-Ile-Trp.
Cyclo-(D-Trp-D-Asp-Pro-DVal-Leu).
Cyclo-(DGlu-A1a-alloDIle-Leu-DTrp).
Cyclo-(D-Trp-D-Asp-Pro-α-(2-thienyl)-D-Gly-Leu).
H-GIy-Asn-Trp-His-Gly-Thr-Ala-Pro-Asp-Trp-Val-Tyr-Phe-Ala-His-Leu-Asp-Ile-Ile-
Trp-OH.
                    \overline{| |}
Cys-Thr-Cys-Asn-Asp-Met-Tyr-Ala-Glu-Glu-Cys-Leu-Asn-
|
\overline{ |}
Phe-Cys-His-Glu-Asp-Val-Ile-Trp.
Glu-Ala-Val-Tyr-Phe-Ala-His-Leu-Asp-Ile-Ile-Trp.
Ac-Leu-Met-Asp-Lys-Glu-Ala-Val-Tyr-Phe-Ala-His-Leu-Asp-Ile-Ile-Trp.
Suc-Asp-Glu-Glu-Ala-Val-Thr-Phe-Ala-His-Leu-Asp-Ile-Ile-Trp.
\overline{| |}
Cys-Val-Tyr-Phe-Cys-His-Asp-Leu-Ile-Ile-Trp.
                    \overline{| |}
Cys-Ser-Cys-Ser-Ser-Leu-Met-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Ser-Ser-Leu-Met-Asp-Lys-Glu-Cys-Val-Tyr-
|
\overline{ |}
Phe-Cys-His-Leu-Thr-γ-methyl-Leu-Ile-Trp.
(DTrp)-Leu-Asp-Ile-Ile-Trp.
Leu-Asp-Ile-Ile-Trp.
Ac-His-Leu-Asp-Ile-Ile-Trp.
Ac-D-His-Leu-Asp-Ile-Ile-Trp.
Ile-Ile-Trp.
Asp-Gly-Gly-Cys-Gly-Cys-(D-Trp)-Leu-Asp-Ile-Ile-Trp.
Asp-GIy-Gly-Cys-Gly-Cys-Phe-(D-Trp)-Leu-Asp-Ile-Ile-Trp
```

Ac-D-Bhg-Leu-Asp-Ile-Ile-Trp, in which Bhg stands for a 10,11-dihydro-5H-dibenzo-[a,d]-cyclohepteneglycine radical,

Ac-D-Bip-Leu-Asp-Ile-Ile-Trp, in which Bip stands for a 4,4′-biphenylalanine radical or a 4-t-butyl-N-[6-(2-hydroxy-ethoxy)-5-(3-methoxy-phenoxy)-4-pyrimidinyl-benzenesulfonamide radical,

a 4-t-butyl-N-[6-(1′,2′-dihydroxy-propyloxy)-5′-(2-methoxy-phenoxy)-2-methoxy-4-pyrimidinyl-benzenesulfonamide radical,

a 4-t-butyl-N-[6′-(2′-hydroxy-ethoxy)-5-(2-methoxy-phenoxy)-2,2′-bipyrimidin-4-yl-benzenylsulfonamide radical,

a 27-O-caffeoylmyricerone radical or a

2(R)-[2-(R)-[2(S)-[[1-(hexahydro-1H-azepinyl)]carbonyl]amino-4-methylpentanoyl]amino-3-[1-methyl-1H-indonyl)]propinonyl]amino-3-(2-pyridyl)propionic acid radical.

As active group W¹, the radionuclides of elements At, Ba, Br, C, F, N, O or P can be mentioned.

Active group (W¹) can, however, also be derived from chemotherapy agents, antibodies, antibody fragments, peptides, carbohydrates, oligonucleotides, PTK blockers, anti-thrombotic agents, growth factor inhibitors, pharmaceutical agents, platelet aggregation inhibitors, anti-inflammatory agents, Ca-antagonists, lipid-lowering agents, or anti-proliferative agents. In this case, one or more, preferably 1 to 10, active groups can each be bonded to the endothelin radical. The bond can optionally also be created via corresponding linkers.

As chemotherapy agents, there can be mentioned by way of example vinblastine, doxorubicin, bleomycin, methotrexate, 5-fluorouracil, 6-thioguanine, cytarabine, cyclophosphamide, and preferably cis-platinum.

As pharmaceutical agents, there can be mentioned by way of example mercaptopurine, N-methyl-formamide, 2-amino-1,3,4-thiadiazole, melphalan, hexamethylmelanine, dichloromethotrexate, mitoguazone, sumarin, bromodeoxyuridine, iododeoxyuridine, semustine, 1-(2-chloroethyl)-3-(2,6-dioxo-3-piperidyl)-1-nitrosourea, N,N′-hexamethylene-bis-acetamide, azacytidine, dibromodulcitol, erwinia-asparaginase, ifosfamide, 2-mercaptoethanesulfonate, teniposide, taxol, 3-deazauridine, soluble Baker's folic acid antagonist, homoharringtonine, cyclocytidine, acivicin, ICRF-187, spiromustine, levamisole, chlorozotocin, aziridinylbenzoquinone, spirogermanium, aclarubicin, pentostatin, PALA, carboplatinum, amsacrine, caracemide, iproplatin, misonidazole, dihydro-5-azacytidine, 4′-deoxy-doxorubicin, menogaril, triciribine phosphate, fazarabine, tiazofurin, teroxirone, ethiofos, N-(2-hydroxyethyl)-2-nitro-1H-imidazole-1-acetamide, mitoxantrone, acodazole, amonafide, fludarabine phosphate, pibenzimol, didemnin B, merbarone, dihydrolene perone, flavone-8-acetic acid, oxantrazole, ipomeanol, trimetrexate, deoxyspergualin, echinomycin, or dideoxycytidine.

Suitable as active groups are, in addition, antithrombotic agents, such as, e.g., heparin, hirudin, low molecular weight heparin, or marcumar; growth factor inhibitors, such as, e.g., anti-PDGF, [e.g., triazolopyrimidine (Trapidil^{(R)})]; platelet aggregation inhibitors, such as, e.g., RGD-peptides, which bind to GP IIb/IIIa receptors, acetylsalicylic acid (Aspirin^{(R)}), dipyridamole, thrombin, clotting cascade inhibitors, such as, e.g., factor VIIa or Xa inhibitors; anti-inflammatory agents, such as, e.g., corticoids or nonsteroidal anti-inflammatory agents; Ca antagonists such as, e.g., verapamil, nifedipine or diltiazem; lipid lowering agents, such as, e.g., simvastatin or probucol; anti-proliferative agents such as, e.g., colchicine, angiopeptin, estradiol or ACE inhibitors (e.g., Ramipril^{(R)}); antisense oligonucleotides; aptamer oligonucleotides; PTK blockers such as, e.g., quercetin, genistein, erbstatin, lavendustin A, herbimycin A or aeroplysinin-1, or synthetic PTK blockers such as, e.g., tyrphostins, S-aryl-benylidene malononitrile compounds or benzylidene malononitrile (BMN) compounds.

Depending on the active group, the linkage of active groups with endothelins is carried out in a way that is known in the art.

Thus, tyrosine kinase inhibitors (PTK blockers) such as tyrphostins can be bonded by, e.g., their phenolic OH groups to the peptides such as endothelin, whereby the latter is first esterified with cyclic anhydrides of aliphatic and aromatic dicarboxylic acids and then amide-linked with the N-terminus of the peptide.

The linkage of cis-platinum to endothelins is done analogously to the methods that are described by Bogdanov et al. (Bioconjugate Chem. 7 (1996) 144-149).

Another aspect of the invention relates to agents that contain an endothelin conjugate that is dissolved, suspended, or emulsified in water and the additives and stabilizers that are commonly used in galenicals. If the endothelin conjugate as an active group carries a complex with a short-lived radioisotope, the corresponding agents are made available as a kit, whereby the endothelin compound that is coupled to the metal-free complexing agent comes in a container. The desired radioisotope is added to the latter immediately before administration.

The agents are preferably administered intravenously. This type of administration thus means that metastases or those lesions that are still very small and cannot be detected diagnostically but will respond especially well to, e.g., therapy with tyrosine kinase inhibitors, antimetabolites, or ionizing beams can be reached in a targeted manner. Thus, e.g., vascular diseases can be healed multifocally.

As shown in Example 5, the substances according to the invention are extremely well suited for being transported in large amounts and over a long period specifically to the wall of a blood vessel via an administration catheter.

The amount that is administered in each case depends on the respective active group and the extent of the deposits. As a rough upper limit, a value can be assumed such as would also be used if pure active ingredient were administered. Owing to the action-enhancing effect as well as the possibility of introducing the active ingredient specifically (via a catheter), in general the necessary dose, however, is far below this upper limit.

If the active group is a radioactive radical, an amount is administered which corresponds to a radiation dose of 1 to 1000 MBq.

Surprisingly, however, the systemic compatibility of highly potent active ingredients is also improved by the binding to the endothelin-receptor-affine substances and endothelin derivatives. Reduction in toxicity for critical organs results despite increased dosage. If necessary, therefore, in many cases the dose can also be increased beyond the extent permissible for the free active ingredient, without an endothelin receptor-mediated incompatibility or incompatibility mediated by the antiproliferative active ingredient occurring.

In addition, relative to DE 43 01 871 and DE 44 25 778, it was found that the endothelin derivatives, surprisingly enough, reach a concentration in the lesions that is sufficient not only for radiotherapy, but also for pharmacotherapy, and have there a dispersion and retention period that are suitable for therapeutic purposes. The extraordinarily quick and efficient uptake of the conjugates upon only brief contact with the arteriosclerotic vessel, as was done in, e.g., administration via a catheter, is especially advantageous.

Owing to their high endothelin receptor affinity, the endothelin conjugates are suitable not only for therapy of cardiovascular diseases, such as, e.g., myocardial ischemia, congestive cardiac failure, cardiac dysrhythmias, unstable angina, myocardial infarction, high blood pressure, arteriosclerosis, and restenosis but also for, e.g., treatment of bronchoconstrictive diseases such as high pulmonary pressure and asthma, neuronal diseases such as cerebral infarction, cerebral vasospasms, and subarachnoid hemorrhages, endocrinal diseases such as pre-eclampsia, renal diseases, vascular diseases such as Buerger's disease, Takayasu's arthritis, Raynaud's phenomenon, micro- and macroangiopathies and all forms of diabetic diseases, neoplastic diseases, especially leiomyoma, pulmonary and prostate carcinomas, gastric mucous membrane injuries, gastrointestinal alterations, endotoxic shock, septicemia as well as bacterial and other inflammations, i.e., all diseases in which the endothelin level as well as the expression of the endothelin receptors are altered (Doherty 1992, J. Med. Chem. 35, 1493-1508, Dashwood et al. 1991, J. Cardiovasc. Pharmacol. 17, Suppl. 7: 458-462, Zeiher et al. 1994, Lancet 344: 1405-1406, Winklers et al. 1993, Biochem. Biophys. Res. Commun. 191: 1081-1088, Ari et al. 1990, Nature 348: 732-735, Goto and Warner 1995, 375: 539-540, Kowala et al. 1995, Am. J. Pathol. 4: 819-827, Douglas et al. 1995, Cardiovascular Research 29: 641-646).

The following examples are used for a more detailed explanation of the subject of the invention, without intending that it be limited to these examples.

### EXAMPLE 1

### a) NHS-Ester of the N′,N′,N′″,N′″-Tetrakis(tert-butyloxycarboxy-methyl)-N″-(hydroxy-carboxy-methyl)-diethylene-triamine

6.178 g (10 mmol) of N′,N′,N′″,N′″-tetrakis(tert-butyloxycarboxy-methyl)-N″-(hydroxy-carboxy-methyl)-diethylene-triamine and 1.15 g (10 mmol) of N-hydroxysuccinimide are dissolved in 90 ml of absolute dimethylformamide. Then, 2.063 g (10 mmol) of dicyclohexylcarbodiimide, dissolved in 10 ml of absolute dimethylformamide, is added in drops to the reaction mixture. It is stirred for 30 minutes at room temperature, filtered, and a 0.1 molar solution of NHS-ester is obtained. The latter is used for the following coupling reactions without further purification.

### b) NH₂-Gly-Phe-(D-Trp)-Leu-Asp-Ile-Ile-Trp-OH

The synthesis of NH₂-Gly-Phe-(D-Trp)-Leu-Asp-Ile-Ile-Trp-OH was carried out by solid-phase synthesis analogously to E. Atherthon and R. C. Sheppard (Solid Phase Peptide Synthesis, A Practical Approach, IRL Press, Oxford, New York, Tokyo, 1989).

### c) N-[N′,N′,N′″,N′″-Tetrakis (hydroxy-carboxy-methyl)-N″-(carboxy-methyl)-diethylin-triamino]-Gly-Phe-(D-Trp)-Leu-Asp-Ile-Ile-Trp-OH

524.6 mg (0.5 mmol) of NH₂-Gly-Phe-(D-Trp)-Leu-Asp-Ile-Ile-Trp-OH (Example 1b) is brought into solution in 100 ml of absolute dimethylformamide in the presence of 202.4 mg (2 mmol) of triethylamine. Under an argon atmosphere, 10 ml of a 0.1 molar solution of the NHS-ester of N′,N′,N′″,N′″-tetrakis(tert-butyloxycarboxy-methyl)-N″-(hydroxy-carboxy-methyl)-diethylene-triamine (produced as described under Example 1a) is added in drops, and the reaction mixture is stirred for 6 hours at room temperature. Then, it is filtered, and the solvent is evaporated in a medium-high vacuum. For cleavage of the tert-butyl ester, the white residue is treated with 150 ml of a mixture of trifluoroacetic acid:anisole:ethanedithiol (95:2.5:2.5). Then, it is concentrated in a medium-high vacuum at room temperature (about 15-20 ml) and poured onto 150 ml of absolute diethyl ether. The white precipitate is suctioned off and purified by chromatography on silica gel RP-18 (eluant: A: water/0.1% trifluoroacetic acid B: acetonitrile/0.1% trifluoroacetic acid; gradient: 0% B to 100% B).

Yield: 80.2 mg (11.3%) of white powder

Molecular weight: Cld: 1424.58 Fnd: 1425 (FAB-MS)

### d) In-111-Complex of N-[N′,N′,N′″,N′″-tetrakis(hydroxycarboxy-methyl)-N″-(carboxy-methyl)-diethylene-triamino]-Gly-Phe-(D-Trp)-Leu-Asp-Ile-Ile-Trp-OH

1 mg of N-[N′,N′,N′″,N′″-tetrakis (hydroxy-carboxy-methyl)-N″-(carboxy-methyl)-diethylin-triamino]-Gly-Phe-(D-Trp)-Leu-Asp-Ile-Ile-Trp-OH (Example 1c) is dissolved in 1 ml of 0.1 molar sodium acetate solution (pH=6) and mixed with 1 mCi of indium-111-trichloride solution (Amersham). The reaction mixture is allowed to stand for 10 minutes at room temperature. The labeling yield is determined by HPLC analysis and is greater than 95%.

### e) Y-90 Complex of N-[N′,N′,N′″,N′″-tetrakis (hydroxy-carboxy-methyl)-N″-(carboxy-methyl)-diethylene-triamino]-Gly-Phe-(D-Trp)-Leu-Asp-Ile-Ile-Trp-OH

1 mg of N-[N′,N′,N′″,N′″-tetrakis (hydroxy-carboxy-methyl)-N″-(carboxy-methyl)-diethylene-triamino]-Gly-Phe-(D-Trp)-Leu-Asp-Ile-Ile-Trp-OH (Example 1c) is dissolved in 1 ml of 0.1 molar sodium acetate solution (pH=6) and mixed with 1 mCi of yttrium-90-trichloride (Amersham). The reaction mixture is allowed to stand for 10 minutes at room temperature. The labeling yield is determined by HPLC analysis and is greater than 94%.

### EXAMPLE 2

### a) N-(8-Amino-1-oxo-octyl)-Phe-(D-Trp)-Leu-Asp-Ile-Ile-Trp-OH

The synthesis of N-(8-amino-1-oxo-octyl)-Phe-(D-Trp)-Leu-Asp-Ile-Ile-Trp-OH was carried out by solid-phase synthesis analogously to E. Atherton and R. C. Sheppard (Solid Phase Peptide Synthesis, A Practical Approach, IRL Press, Oxford, New York, Tokyo, 1989).

### b) N-[N′,N′,N′″,N′″-Tetrakis (hydroxy-carboxy-methyl)-N″-(carboxy-methyl)-diethylin-triamino]-[(8-amino-1-oxo-octyl)-Phe-(D-Trp)-Leu-Asp-Ile-Ile-Trp-OH]

566.7 mg (0.5 mmol) of (8-amino-1-oxo-octyl)-Phe-(D-Trp)-Leu-Asp-Ile-Ile-Trp-OH (Example 2a) is brought into solution in 100 ml of absolute dimethylformamide in the presence of 202.4 mg (2 mmol) of triethylamine. Under an argon atmosphere, 10 ml of a 0.1 molar solution of the NHS-ester of N′,N′,N′″,N′″-tetrakis(tert-butyloxycarboxy-methyl)-N″-(hydroxy-carboxy-methyl)-diethylene-triamine (produced as described under Example 1a) is added in drops, and the reaction mixture is stirred for 6 hours at room temperature. Then, it is filtered, and the solvent is evaporated at room temperature in a medium-high vacuum. For cleavage of the tert-butyl ester, the white residue is treated with 150 ml of a mixture that consists of trifluoroacetic acid:anisole:ethanedithiol (95:2.5:2.5). Then, it is concentrated in a medium-high vacuum at room temperature (about 15-20 mol) and poured onto 150 ml of absolute diethyl ether. The white precipitate is suctioned off and purified by chromatography on silica gel RP-18 (eluant: A: water/0.1% of trifluoroacetic acid B: acetonitrile/0.1% trifluoroacetic acid; gradient: 0% B to 100% B).

| | |
|---|---|
| Yield: | 135.2 mg (17.9%) of white powder |
| Molecular weight: | |
| Cld: | 1508.74 |
| Fnd: | 1509 (FAB-MS) |

c) In-111 Complex of N-[N′,N′,N′″,N′″-tetrakis-(hydroxycarboxy-methyl)-N″-(carboxy-methyl)-diethylene-triamino]-[(8-amino-1-oxo-octyl)-Phe-(D-Trp)-Leu-Asp-Ile-Ile-Trp-OH]

1 mg of N-[N′,N′,N′″,N′″-tetrakis-(hydroxycarboxy-methyl)-N″-(carboxy-methyl)-diethylene-triamino]-[(8-amino-1-oxo-octyl)-Phe-(D-Trp)-Leu-Asp-Ile-Ile-Trp-OH] (Example 2b) is dissolved in 1 ml of 0.1 molar sodium acetate solution (pH=6) and mixed with 1 mCi of indium-111-trichloride solution (Amersham). The reaction mixture is allowed to stand for 10 minutes at room temperature. The labeling yield is determined by HPLC analysis and is greater than 94%.

### d) Y-90 Complex of N-[N′,N′,N′″,N′″-tetrakis-(hydroxycarboxy-methyl)-N″-(carboxy-methyl)-diethylene-triamino]-[(8-amino-1-oxo-octyl)-Phe-(D-Trp)-Leu-Asp-Ile-Ile-Trp-OH]

1 mg of N-[N′,N′,N′″,N′″-tetrakis-(hydroxycarboxy-methyl)-N″-(carboxy-methyl)-diethylene-triamino]-[(8-amino-1-oxo-octyl)-Phe-(D-Trp)-Leu-Asp-Ile-Ile-Trp-OH] (Example 2b) is dissolved in 1 ml of 0.1 molar sodium acetate solution (pH=6) and mixed with 1 mCi of yttrium-90 trichloride solution (Amersham). The reaction mixture is allowed to stand for 10 minutes at room temperature. The labeling yield is determined by HPLC analysis and is greater than 97%.

### EXAMPLE 3

### a) NH₂-Asp-Gly-Gly-Cys-Gly-Cys-Phe-(D-Trp)-Leu-Asp-Ile-Ile-Trp-OH

The synthesis of NH₂-Asp-Gly-Gly-Cys-Gly-Cys-Phe-(D-Trp)-Leu-Asp-Ile-Ile-Trp-OH was carried out by solid-phase synthesis analogously to E. Atherton and R. C. Sheppard (Solid Phase Peptide Synthesis, A Practical Approach, IRL Press, Oxford, New York, Tokyo, 1989).

### b) Rhenium-186 Complex of NH₂-Asp-Gly-Gly-Cys-Gly-Cys-Phe-(D-Trp)-Leu-Asp-Ile-Ile-Trp-OH

1 mg of NH₂-Asp-Gly-Gly-Cys-Gly-Cys-Phe-(D-Trp)-Leu-Asp-Ile-Ile-Trp-OH in 600 μl of phosphate buffer (Na₂HPO₄, 0.5 mol/l, pH=8.5) is mixed with 100 μl of a 0.15 molar trisodium citrate dihydrate solution, 500 μCi of 186-perrhenate solution and finally with 5 ηl of a 0.2 molar tin(II) chloride-dihydrate solution. It is incubated for 10 minutes at room temperature. The analysis of the labeling is carried out using HPLC.

### EXAMPLE 4

### a) NH₂-Asp-Gly-Gly-Cys-Gly-Cys-(D-Trp)-Leu-Asp-Ile-Ile-Trp-OH

The synthesis of NH₂-Asp-Gly-Gly-Cys-Gly-Cys-(D-Trp)-Leu-Asp-Ile-Ile-Trp-OH was carried out by solid-phase synthesis analogously to E. Atherton and R. C. Sheppard (Solid-Phase Peptide Synthesis, A Practical Approach, IRL Press, Oxford, New York, Tokyo, 1989).

### b) Rhenium-186-Complex of NH₂-Asp-Gly-Gly-Cys-Gly-Cys-(D-Trp)-Leu-Asp-Ile-Ile-Trp-OH

1 mg of NH₂-Asp-Gly-Gly-Cys-Gly-Cys-(D-Trp)-Leu-Asp-Ile-Ile-Trp-OH in 600 μl of phosphate buffer (Na₂HPO₄, 0.5 mol/l, pH=8.5) is mixed with 100 μl of a 0.15 molar trisodium citrate dihydrate solution, 500 μCi of 186-perrhenate solution and finally with 5 μl of a 0.2 molar tin(II) chloride-dihydrate solution. It is incubated for 10 minutes at room temperature. The analysis of the labeling is done using HPLC.

### EXAMPLE 5

### a) ^{99m}Tc Complex of Asp-Gly-Gly-Cys-Gly-Cys-Phe-(D-Trp)-Leu-Asp-Ile-Ile-Trp

0.5 mg of Asp-Gly-Gly-Cys-Gly-Cys-Phe-(D-Trp)-Leu-Asp-Ile-Ile-Trp [produced as described in Example 3a)] in 300 μl of phosphate buffer (Na₂HPO₄, 0.5 mol/l, pH 8.5) is mixed with 50 μl of a 0.15 molar trisodium citrate dihydrate solution and 2.5 μl of a 0.2 molar tin(II) chloride dihydrate solution. The reaction mixture is mixed with a pertechnetate solution (0.4 to 0.9 mCi) from an Mo-99/Tc-99m generator, incubated for 10 minutes at room temperature. The analysis of the labeling is done via HPLC.

### b) Local Application of the Tc-99m Complex of NH₂-Asp-Gly-Gly-Cys-Gly-Cys-Phe-(D-Trp)-Leu-Asp-Ile-Ile-Trp-OH as well as Tc-99m-pertechnetate in the Common Carotid Artery of White New Zealand Rabbits

In anesthetized white New Zealand rabbits (3.5 kg), the right common carotid artery was opened up. A 2 F balloon catheter (from the Baxter Company) was inserted cranially through a cut, and a vascular area approximately 5 cm long was denuded twice with 0.9% saline after the catheter was inflated. Then, an administration catheter (coronary perfusion/infusion catheter, dispatch 3.0, Baxter Company) was fed to the above-denuded area. 0.9 ml of an activity of either 7.4 MBq of Tc-99m-NH₂-Asp-Gly-Gly-Cys-Gly-Cys-Phe-(D-Trp)-Leu-Asp-Ile-Ile-Trp-OH [produced as described in 5a)] or Tc-99m pertechnetate was administered locally. Then, the catheter was removed, and the blood flow after closure of the common carotid artery dextra was restored using a suture. Dynamic scintigrams were prepared over a period of 1 hour with the aid of a commercial gamma camera. Then, the animals were sacrificed, both carotids were removed, and an autoradiography was prepared.

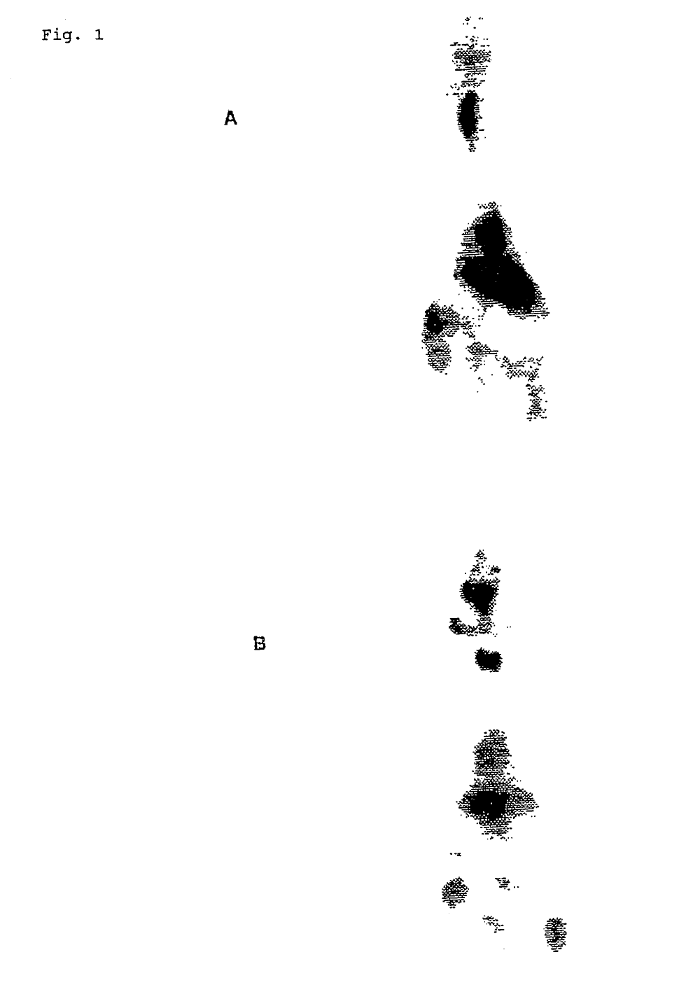

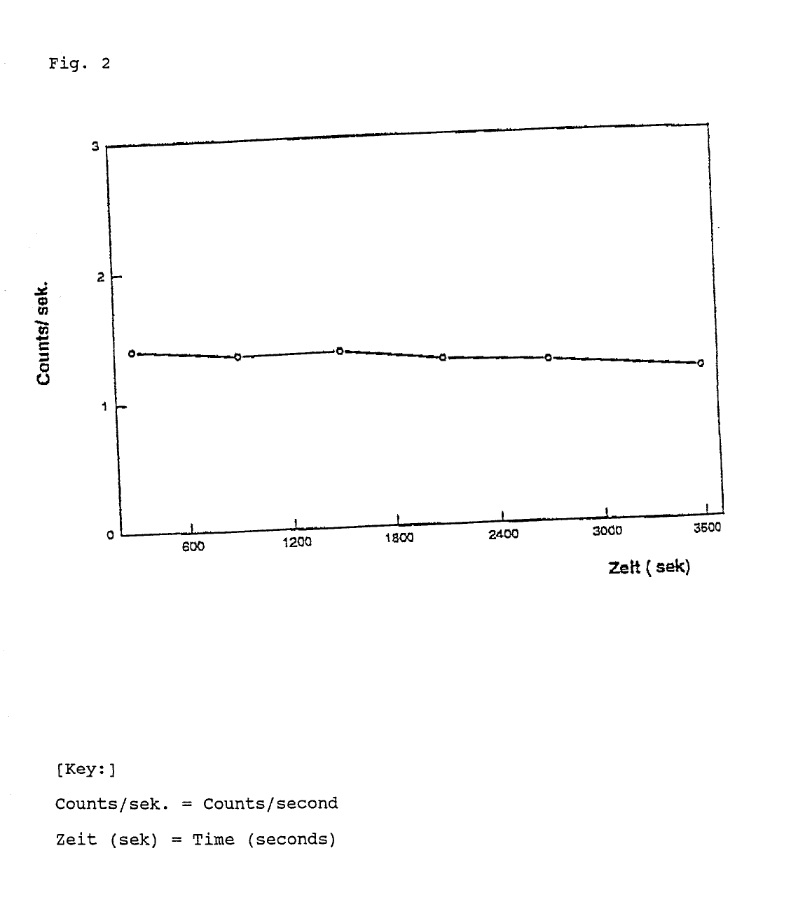

In the case of Tc-99m-NH₂-Asp-Gly-Gly-Cys-Gly-Cys-Phe-(D-Trp)-Leu-Asp-Ile-Ile-Trp-OH, it was possible to transport about 5% of the injected dose locally to the denuded artery. The administered activity decreased only insignificantly over the examination period. However, local administration of Tc-99m pertechnetate was not successful, since the overall locally administered activity was flushed from the vessel directly after the blood flow was regenerated (see FIGS. 1 and 2).

FIG. 1 shows an anterior summation scintigram of the dynamic study 0-1 hour after local administration of the Tc-99m complex of NH₂-Asp-Gly-Gly-Cys-Gly-Cys-Phe-(D-Trp)-Leu-Asp-Ile-Ile-Trp-OH (Image A) as well as of Tc-99m-pertechnetate (Image B). While the locally administered Tc-99m-NH₂-Asp-Gly-Gly-Cys-Gly-Cys-Phe-(D-Trp)-Leu-Asp-Ile-Ile-Trp-OH remains in place during the examination period of 1 hour after the blood flow is restored at the administration site (A, arrow), Tc-99m-pertechnetate (Image B) is flushed from the vessel wall immediately after the blood flow is restored and accumulates in the salivary glands as well as the thyroid.

FIG. 2 shows the course of the activity (cpm/s) of Tc-99m-NH₂-Asp-Gly-Gly-Cys-Gly-Cys-Phe-(D-Trp)-Leu-Asp-Ile-Ile-Trp-OH in the right common carotid artery after local administration over time. The activity was recorded over a period of 1 hour after local administration by a dynamic study. During the examination period, the locally administered amount of Tc-99m-NH₂-Asp-Gly-Gly-Cys-Gly-Cys-Phe-(D-Trp)-Leu-Asp-Ile-Ile-Trp-OH decreased only marginally.

### EXAMPLE 6

### In Vivo and In Vitro Concentration of the ^{99m}Tc-Complex of Asp-Gly-Gly-Cys-Gly-Cys-Phe-(D-Trp)-Leu-Asp-Ile-Ile-Trp in WHHL Rabbits

2 mCi (1 ml) of the Asp-Gly-Gly-Cys-Gly-Cys-Phe-(D-Trp)-Leu-Asp-Ile-Ile-Trp ^{99m}Tc-complex that is produced according to 5a) is administered to an anesthetized WHHL rabbit (Rompun/Ketavet 1:2) via an ear vein. WHHL rabbits have a high LDL level in the blood because of a deficient or defective LDL receptor and therefore create spontaneously arteriosclerotic vascular alterations. During the test period of 5 hours after the administration, static images of various exposure times and of various positions were produced with a gamma camera (Elcint SP4HR). 5 hours after the administration, the rabbit was sacrificed, and both an autoradiography of the aorta and a Sudan III coloring were carried out. It was possible to visualize the arteriosclerotic plaque in the area of the aortic arch of WHHL rabbits for 10 minutes p.i. in vivo. The autoradiography that was then performed yielded an accumulation of 3930 cpm/mm² arteriosclerotic lesions and an accumulation of 380 cpm/mm² in the macroscopically unaltered aorta. The concentration factor between normal and arteriosclerotic wall areas was 14.

### EXAMPLE 7

Linkage of Erbstatin with H₂N-Gly-Phe-(DTrp)-Leu-Asp-Ile-Ile-Trp-OH

1.79 g (0.01 mol) of erbstatin is dissolved in 100 ml of methylene chloride, a nitrogen atmosphere is prepared, and 1 g (0.01 mol) of succinic acid anhydride as well as 1.74 ml (0.01 mol) of diisopropylethylamine are added and stirred overnight at room temperature. 1.15 g (0.01 mol) of N-hydroxysuccinimide (NHS) in solid form is added to this solution, and after its dissolution, a solution of 2.06 g (0.01 mol) of dicyclohexylcarbodiimide (DCCI) in 20 ml of methylene chloride is added in drops. Again, it is stirred overnight at room temperature. For working-up, the precipitated dicyclohexylurea is filtered off, the filtrate is washed twice with 1% citric acid and once with saturated sodium bicarbonate solution, dried with magnesium sulfate and concentrated by evaporation. The residue is dissolved in a little methylene chloride, and the residual precipitated dicyclohexylurea is filtered off. The filtrate is concentrated by evaporation, and the residue is taken up in DMF. 10.5 g of (0.01 mol) of H₂N-Gly-Phe-(DTrp)-Leu-Asp-Ile-Ile-Trp-OH is added, and it is stirred overnight at room temperature. The solution is concentrated by evaporation in a medium-high vacuum, and the residue is chromatographed on silica gel with the mobile solvent system of methylene chloride/methanol (gradient of 3% to 20% methanol).

| | |
|---|---|
| Result: | 3.14 g (24% of theory) of light yellow crystals |
| Molecular weight: | |
| Cld: | 1310.47 |
| Fnd: | 1310 m/e (FAB-MS) |
| Elementary analysis: | |
| Cld: | C 62.3% H 6.4% N 11.8% O 19.5% |
| Fnd: | C 61.8% H 6.3% N 11.4% |

### EXAMPLE 8

### 2-Acetyloxybenzoyl-Gly-Phe-(D-Trp)-Leu-Asp-Ile-Ile-Trp-OH

524.6 mg (0.5 mmol) of NH₂-Gly-Phe-(D-Trp)-Leu-Asp-Ile-Ile-Trp-OH (Example 1b) is brought into solution in 100 ml of absolute DMF in the presence of 202.4 mg (2 mmol) of triethylamine. Under a nitrogen atmosphere, a solution of 1.39 g of acetylsalicylic acid-N-hydroxysuccinimide ester (5 mmol) in 10 ml of DMF is added in drops, and it is allowed to stir overnight at room temperature. The reaction mixture is concentrated by evaporation in a medium-high vacuum, mixed with water and stirred for 30 minutes. Then, the water and easily volatilized components are removed in a medium-high vacuum, and the residue is chromatographed immediately on RP-18 silica gel (eluant A: water; eluant B: acetonitrile; gradient 0% B to 100% B).

| | |
|---|---|
| Yield: | 86.3 mg (= 14.3% of theory) of a white powder |
| Molecular weight: | |
| Cld: | 1212.35 |
| Fnd: | 1212 (FAB-MS) |

### EXAMPLE 9

### a) [21-O-(6α,9-Difluoro-11β,21-dihydroxy-16α-methyl-1,4-pregnadiene-3,20-dionyl)]-2-carboxy-ethylcarboxylic Acid

3.945 (10 mmol) of diflucortolone and 1.0 g (10 mmol) of succinic acid anhydride are refluxed under an argon atmosphere in 20 ml of absolute pyridine for 1 hour. The cooled reaction mixture is poured onto a mixture of sulfuric acid/ice water, and the solid is filtered off. It is recrystallized from acetone/n-hexane.

| Yield: | 2.42 g (48.9%) of white powder | | | |
|---|---|---|---|---|
| Elementary analysis: | | | | |
| Cld: | C 63.15 | H 6.52 | O 22.65 | F 7.68 |
| Fnd: | C 62.95 | H 6.76 | | F 7.53 |

### b) [21-O-(6α,9-Difluoro-11β,21-dihydroxy-16α-methyl-1,4-pregnadiene-3,20-dionyl)]-2-carboxy-ethylcarboxylic Acid-N-hydroxysuccinimide Ester

4.95 g (10 mmol) of the diflucortolone derivative that is described under Example 9a) and 1.15 g (10 mmol) of N-hydroxysuccinimide are dissolved in 90 ml of absolute dimethylformamide. Then, 2.063 g (10 mmol) of dicyclohexylcarbodiimide, dissolved in 10 ml of absolute dimethylformamide, is added in drops to the reaction mixture. It is stirred for 45 minutes at room temperature, filtered, and a 0.1 molar solution of the NHS ester is obtained. The latter is used for the following coupling reactions without further purification.

### c) {[21-O-(6α,9-Difluoro-11β,21-dihydroxy-16α-methyl-1,4-pregnadiene-3,20-dionyl)]-2-carboxy-ethylcarboxy}Gly-Phe-(D-Trp)-Leu-Asp-Ile-Ile-Trp-OH

524.6 mg (0.5 mmol) of NH₂-Gly-Phe-(D-Trp)-Leu-Asp-Ile-Ile-Trp-OH (Example 1b) is brought into solution in 100 ml of absolute dimethylformamide in the presence of 202.4 mg (2 mmol) of triethylamine. Under an argon atmosphere, 10 ml of a 0.1 molar solution of the NHS-ester (Example 9b) is added in drops, and the reaction mixture is stirred for 14 hours at room temperature. Then, it is filtered, and the solvent is evaporated in a medium-high vacuum. The residue is purified by chromatography on RP-18 (eluant: A: water, B: acetonitrile; gradient: 0% B to 100% B).

| | |
|---|---|
| Yield: | 72.3 mg (9.5%) of white powder |
| Molecular weight: | |
| Cld: | 1525.76 |
| Fnd: | 1526 (FAB-MS) |

1. Use of the compounds of general formula (I)
E-Wₙ (I)
in which
E stands for a radical that binds endothelin receptors and is derived from endothelins, endothelin analogs, endothelin derivatives, endothelin partial sequences, and endothelin antagonists, and
W stands for an active group that is a radionuclide or that is derived from a chemotherapy agent, a complex with a radioactive metal isotope, an antibody, antibody fragment, peptide, carbohydrate, oligonucleotide, PTK blocker, antithrombotic agent, clotting cascade inhibitor, hormone, growth factor inhibitor, pharmaceutical agent, platelet aggregation inhibitor, anti-inflammatory agent, Ca-antagonist, lipid-lowering agent, or an antiproliferative agent, and
n stands for numbers 1 to 100, preferably 1 to 10, as therapeutic agents.

2. Use of the compounds of general formula E-Wₙ, in which E, W, and n have the meaning that is indicated in claim 1 as therapeutic agents for treating vascular diseases.

3. Use according to claim 1 or 2, in which the radical that binds the endothelin receptor has the structure
```
                    \overline{| |}
Cys-Ser-Cys-Ser-Ser-Leu-Met-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Ser-Ser-Trp-Leu-Asp-Lys-Glu-Cys-Val-Tyr-
\overline{ |}
Phe-Cys-His-Leu-Asp-Ile-Ile-Trp.
                    \overline{| |}
Cys-Thr-Cys-Phe-Thr-Tyr-Lys-Asp-Lys-Glu-Cys-Val-Tyr-
|
\overline{ |}
Tyr-Cys-His-Leu-Asp-Ile-Ile-Trp.
                    \overline{| |}
Cys-Ser-Cys-Ser-Ser-Trp-Leu-Asp-Lys-Glu-Cys-Val-Tyr-
|
\overline{ |}
Phe-Cys-His-Leu-Asp-Ile-Ile-Trp.
                    \overline{| |}
Cys-Thr-Cys-Phe-Thr-Tyr-Lys-Asp-Lys-Glu-Cys-Val-Tyr-
|
\overline{ |}
Tyr-Cys-His-Leu-Asp-Ile-Ile-Trp.
Cys-Ser-Ala-Ser-Ser-Leu-Met-Asp-Lys-Glu-Ala-Val-Tyr-
|
\overline{ |}
Phe-Cys-His-Leu-Asp-Ile-Ile-Trp,
                    \overline{| |}
Cys-Ser-Cys-Asn-Ser-Trp-Leu-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Lys-Asp-Met-Thr-Asp-Lys-Glu-Cys-Leu-Asn-
|
\overline{ |}
Phe-Cys-His-Gln-Asp-Val-Ile-Trp.
                    \overline{| |}
Ala-Ser-Cys-Ser-Ser-Leu-Met-Asp-Lys-Glu-Cys-Val-Tyr-
Phe-Ala-His-Leu-Asp-Ile-Ile-Trp.
Ala-Ser-Ala-Ser-Ser-Leu-Met-Asp-Lys-Glu-Ala-Val-Tyr-Phe-Ala-His-Leu-Asp-
Ile-Ile-Trp.
Cys-Ser-Cys-Ser-Ser-Trp-Leu-Asp-Lys-Glu-Ala-Val-Tyr-Phe-Ala-His-Leu-Asp-
Ile-Ile-Trp.
\overline{| |}
Cys-Val-Tyr-Phe-Cys-His-Leu-Asp-Ile-Ile-Trp.
N-Acetyl-Leu-Met-Asp-Lys-Glu-Ala-Val-Tyr-Phe-Ala-His-Leu-Asp-Ile-Ile-Trp.
His-Leu-Asp-Ile-Ile-Trp.
(DTrp)-Leu-Asp-Ile-Ile-Trp.
Cyclo-(DTrp-DAsp-Pro-DVal-Leu).
Cyclo-(DGlu-Ala-alloDIle-Leu-DTrp).
Cyc1o-(D-Trp-D-Asp-Pro-α-(2-thienyl)-D-Gly-Leu).
H-Gly-Asn-Trp-His-Gly-Thr-Ala-Pro-Asp-Trp-Val-Tyr-Phe-Ala-His-Leu-Asp-Ile-
Ile-Trp-OH.
                    \overline{| |}
Cys-Thr-Cys-Asn-Asp-Met-Tyr-Ala-Glu-Glu-Cys-Leu-Asn-
|
\overline{ |}
Phe-Cys-His-Glu-Asp-Val-Ile-Trp.
Glu-Ala-Val-Tyr-Phe-Ala-His-Leu-Asp-Ile-Ile-Trp.
Ac-Leu-Met-Asp-Lys-Glu-Ala-Val-Tyr-Phe-Ala-His-Leu-Asp-Ile-Ile-Trp.
Suc-Asp-Glu-Glu-Ala-Val-Thr-Phe-Ala-His-Leu-Asp-Ile-Ile-Trp.
\overline{| |}
Cys-Val-Tyr-Phe-Cys-His-Asp-Leu-Ile-Ile-Trp.
                    \overline{| |}
Cys-Ser-Cys-Ser-Ser-Leu-Met-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Ser-Ser-Leu-Met-Asp-Lys-Glu-Cys-Val-Tyr-
|
\overline{ |}
Phe-Cys-His-Leu-Thr-γ-methyl-Leu-Ile-Trp
```
or is
a 4-t-butyl-N-[6-(2-hydroxy-ethoxy)-5-(3-methoxy-phenoxy)-4-pyrimidinyl-benzenesulfonamide radical,
a 4-t-butyl-N-[6-(1′,2′-dihydroxy-propyloxy)-5′-(2-methoxy-phenoxy)-2-methoxy-4-pyrimidinyl-benzenesulfonamide radical,
a 4-t-butyl-N-[6′-(2′-hydroxy-ethoxy)-5-(2-methoxy-phenoxy)-2,2′-bipyrimidin-4-yl-benzenylsulfonamide radical,
a 27-O-caffeoylmyricerone radical, or
a 2(R)-[2-(R)-[2(S)-[[1-(hexahydro-1H-azepinyl)]carbonyl]amino-4-methylpentanoyl]amino-3-[1-methyl-1H-indonyl)]propinonyl]amino-3-(2-pyridyl)propionic acid radical.

4. Use according to claim 1 or 2, in which the radical that binds the endothelin receptor has the structure
Leu-Asp-Ile-Ile-Trp,
Ac-His-Leu-Asp-Ile-Ile-Trp,
Ac-D-His-Leu-Asp-Ile-Ile-Trp,
Ile-Ile-Trp,
Asp-Gly-Gly-Cys-Gly-Cys-(D-Trp)-Leu-Asp-Ile-Ile-
Trp,
Ac-D-Bhg-Leu-Asp-Ile-Ile-Trp,
in which Bhg stands for a 10,11-dihydro-5H-dibenzo-[a,d]-cyclohepteneglycine radical,
Ac-D-Bip-Leu-Asp-Ile-Ile-Trp, in which Bip stands for a 4,4′-biphenylalanine radical, or the structure
Asp-Gly-Gly-Cys-Gly-Cys-Phe-(D-Trp)-Leu-Asp-Ile-Ile-Trp.

5. Use according to one of claims 1 to 4, in which the active group contains an alpha-, beta- and/or gamma-radiator, positron radiator, Auger electron radiator, x-ray radiator and/or a fluorescence radiator.

6. Use according to claim 5, in which the active group contains a radionuclide of the elements Ag, As, At, Au, Ba, Bi, Br, C, Co, Cr, Cu, F, Fe, Ga, Gd, Hg, Ho, I, In, Ir, Lu, Mn, N, O, P, Pb, Pd, Pm, Re, Rh, Ru, Sb, Sc, Se, Sm, Sn, Tb, Tc or Y.

7. Use according to claim 5, in which the active groups are derived from a metal complex of a radionuclide of the elements Ag, As, Au, Bi, Cu, Ga, Gd, Hg, Ho, In, Ir, Lu, Pb, Pd, Pm, Pr, Re, Rh, Ru, Sb, Sc, Se, Sm, Sn, Tb, Tc or Y.

8. Use according to one of claims 5 to 7, in which the radionuclide is ¹⁸⁸Re, ⁹⁰Y or ¹¹¹In.

9. Compounds of general formula (II)
E-W¹ₙ (II)
in which
E stands for a radical that binds endothelin receptors and is derived from endothelins, endothelin analogs, endothelin derivatives, endothelin partial sequences, and endothelin antagonists, and
W¹ stands for an active group that contains a radionuclide of the elements At, Ba, Br, C, F, N, O or P or that is derived from a chemotherapy agent, an antibody, antibody fragment, peptide, carbohydrate, oligonucleotide, PTK blocker, antithrombotic agent, growth factor inhibitor, pharmaceutical agent, hormone, platelet aggregation inhibitor, anti-inflammatory agent, Ca-antagonist, lipid-lowering agent, or an antiproliferative agent, and
n stands for numbers 1 to 100, preferably 1 to 10.

10. Compounds according to claim 9, in which the radical that binds the endothelin receptor has the structure
```
                    \overline{| |}
Cys-Ser-Cys-Ser-Ser-Leu-Met-Asp-Lys-Glu-Cys-Val-Tyr-
|
\overline{ |}
Phe-Cys-His-Leu-Asp-Ile-lle-Trp.
                    \overline{| |}
Cys-Ser-Cys-Ser-Ser-Trp-Leu-Asp-Lys-Glu-Cys-Val-Tyr-
|
\overline{ |}
Phe-Cys-His-Leu-Asp-Ile-Ile-Trp.
                    \overline{| |}
Cys-Thr-Cys-Phe-Thr-Tyr-Lys-Asp-Lys-Glu-Cys-Val-Tyr-
|
\overline{ |}
Tyr-Cys-His-Leu-Asp-Ile-Ile-Trp.
                    \overline{| |}
Cys-Ser-Cys-Ser-Ser-Trp-Leu-Asp-Lys-Glu-Cys-Val-Tyr-
|
\overline{ |}
Phe-Cys-His-Leu-Asp-Ile-Ile-Trp.
                    \overline{| |}
Cys-Thr-Cys-Phe-thr-Tyr-Lys-Asp-Lys-Glu-Ala-Val-Tyr-
|
\overline{ |}
Tyr-Cys-His-Leu-Asp-Ile-Ile-Trp,
Cys-Ser-Ala-Ser-Ser-Leu-Met-Asp-Lys-Glu-Ala-Val-Tyr-
|
\overline{ |}
Phe-Cys-His-Leu-Asp-Ile-Ile-Trp.
                    \overline{| |}
Cys-Ser-Cys-Asn-ser-Trp-Leu-Asp-Lys-Glu-Cys-Val-Tyr-
|
\overline{ |}
Phe-Cys-His-Leu-Asp-Ile-Ile-Trp.
                    \overline{| |}
Cys-Ser-Cys-Lys-Asp-Met-Thr-Asp-Lys-Glu-Cys-Leu-Asn-
|
\overline{ |}
Phe-Cys-His-Gln-Asp-Val-Ile-Trp.
                    \overline{| |}
Ala-Ser-Cys-Ser-Ser-Leu-Met-Asp-Lys-Glu-Cys-Val-Tyr-
Phe-Ala-His-Leu-Asp-Ile-Ile-Trp.
Ala-Ser-Ala-Ser-Ser-Leu-Met-Asp-Lys-Glu-Ala-Val-Tyr-Phe-Ala-His-Leu-Asp-
Ile-Ile-Trp,
Cys-Ser-Cys-Ser-Ser-Trp-Leu-Asp-Lys-Glu-Ala-Val-Tyr-Phe-Ala-His-Leu-Asp-
Ile-Ile-Trp.
\overline{| |}
Cys-Val-Tyr-Phe-Cys-His-Leu-Asp-Ile-Ile-Trp.
N-Acetyl-Leu-Met-Asp-Lys-Glu-Ala-Val-Tyr-Phe-Ala-His-Leu-Asp-Ile-Ile-Trp.
His-Leu-Asp-Ile-Ile-Trp.
(DTrp)-Leu-Asp-Ile-Ile-Trp.
Cyclo-(DTrp-DAsp-Pro-DVa1-Leu),
Cyclo-(DGlu-A1a-alloDIle-Leu-DTrp),
Cyclo(D-Trp-D-Asp-Pro-α-(2-thienyl)-D-Gly-Leu).
H-Gly-Asn-Trp-His-Gly-A1a-Pro-Asp-Trp-Val-Tyr-Phe-Ala-His-Leu-Asp-Ile-
Ile-Trp-OH.
                    \overline{| |}
Cys-Thr-Cys-Asn-Asp-Met-Tyr-Ala-Glu-Glu-Cys-Leu-Asn-
|
\overline{ |}
Phe-Cys-His-Glu-Asp-Val-Ile-Trp,
Glu-A1a-Va1-Tyr-Phe-Ala-His-Leu-Asp-Ile-Ile-Trp,
Ac-Leu-Met-Asp-Lys-Glu-Ala-Val-Tyr-Phe-Ala-His-Leu-Asp-Ile-Ile-Trp.
Suc-Asp-Glu-Glu-Ala-Val-Thr-Phe-Ala-His-Leu-Asp-Ile-Ile-Trp.
\overline{| |}
Cys-Val-Tyr-Phe-Cys-His-Asp-Leu-Ile-Ile-Trp.
                    \overline{| |}
Cys-Ser-Cys-Ser-Ser-Leu-Met-Asp-Lys-Glu-Cys-Val-Tyr-
|
\overline{ |}
Phe-Cys-His-Leu-Asp-Ile-IIe-Trp.
                    \overline{| |}
Cys-Ser-Cys-Ser-Ser-Leu-Met-Asp-Lys-Glu-Cys-Val-Tyr-
|
\overline{ |}
Phe-Cys-His-Leu-Thr-γ-methyl-Leu-Ile-Trp.
Leu-Asp-Ile-Ile-Trp.
Ac-His-Leu-Asp-Ile-Ile-Trp.
Ac-D-His-Leu-Asp-Ile-Ile-Trp.
Ile-Ile-Trp.
Asp-Gly-Gly-Cys-Gly-Cys-(D-Trp)-Leu-Asp-Ile-Ile-Trp.
```
Ac-D-Bhg-Leu-Asp-Ile-Ile-Trp, in which Bhg stands for a 10,11-dihydro-5H-dibenzo-[a,d]-cycloheptenylglycine radical,
Ac-D-Bip-Leu-Asp-Ile-Ile-Trp, in which Bip stands for a 4,4′-biphenylalanine radical or the structure
Asp-Gly-Gly-Cys-Gly-Cys-Phe-(D-Trp)-Leu-Asp-Ile-Ile-Trp or is
a 4-t-butyl-N-[6-(2-hydroxy-ethoxy)-5-(3-methoxy-phenoxy)-4-pyrimidinyl-benzenesulfonamide radical,
a 4-t-butyl-N-[6-(1′,2′-dihydroxy-propyloxy)-5′-(2-methoxy-phenoxy)-2-methoxy-4-pyrimidinyl-benzenesulfonamide radical,
a 4-t-butyl-N-[6′-(2′-hydroxy-ethoxy)-5-(2-methoxy-phenoxy)-2,2′-bipyrimidin-4-yl-benzenylsulfonamide radical,
a 27-O-caffeoylmyricerone radical, or
a 2(R)-[2-(R)-[2(S)-[[1-(hexahydro-1H-azepinyl)]carbonyl]amino-4-methylpentanoyl]amino-3-[1-methyl-1H-indonyl)]propinonyl]amino-3-(2-pyridyl)propionic acid radical.

11. Compound according to claim 9 or 10, in which the active group contains a radionuclide of the elements At, Ba, Br, C, F, N, O or P.

12. Compound according to claim 9 or 10, in which the active group is vinblastine, doxorubicin, bleomycin, methotrexate, 5-fluorouracil, 6-thioguanine, cytarabine, cyclophosphamide or a cis-platinum radical.

13. Compound according to claim 9 or 10, in which the active group is derived from a quercetin, genistein, erbstatin, lavendustin A, herbimycin A, aeroplysinin-1-tyrphostin-, S-aryl-benylidene malononitrile or benzylidene malononitrile radical.

14. Compound according to claim 9 or 10, in which the active group is derived from a mercaptopurine, N-methyl-formamide, 2-amino-1,3,4-thiadiazole, melphalan, hexamethylmelanine, dichloromethotrexate, mitoguazone, sumarin, bromodeoxyuridine, iododeoxyuridine, semustine, 1-(2-chloroethyl)-3-(2,6-dioxo-3-piperidyl)-1-nitrosourea, N,N′-hexamethylene-bis-acetamide, azacytidine, dibromodulcitol, erwinia-asparaginase, ifosfamide, 2-mercaptoethanesulfonate, teniposide, taxol, 3-deazauridine, folic acid antagonist, homoharringtonine, cyclocytidine, acivicin, ICRF-187, spiromustine, levamisole, chlorozotocin, aziridinylbenzoquinone, spirogermanium, aclarubicin, pentostatin, PALA, carboplatinum, amsacrine, caracemide, iproplatin, misonidazole, dihydro-5-azacytidine, 4′-deoxy-doxorubicin, menogaril, triciribine phosphate, fazarabine, tiazofurin, teroxirone, ethiofos, N-(2-hydroxyethyl)-2-nitro-1H-imidazole-1-acetamide, mitoxantrone, acodazole, amonafide, fludarabine phosphate, pibenzimol, didemnin B, merbarone, dihydrolene perone, flavone-8-acetic acid, oxantrazole, ipomeanol, trimetrexate, deoxyspergualin, echinomycin or a dideoxycytidine radical.

15. Compound according to claim 9 or 10, in which the active group is derived from an anti-PDGF or a triazolopyrimidine.

16. Compound according to claim 9 or 10, in which the active group is derived from an RGD-peptide, which binds to GP IIb/IIIa receptors, from an acetylsalicylic acid, dipyridamole or thrombin radical.

17. Compound according to claim 9 or 10, in which the active group is derived from heparin, hirudin, low molecular weight heparin or marcumar.

18. Compound according to claim 9 or 10, in which the active group is derived from factor VIIa or Xa inhibitors.

19. Compound according to claim 9 or 10, in which the active group is derived from a corticoid or a nonsteroidal anti-inflammatory agent.

20. Compound according to claim 9 or 10, in which the active group is derived from colchicine, angiopeptin, estradiol or an ACE inhibitor.

21. Compound according to claim 9 or 10, in which the active group is derived from verapamil, nifedipine or diltiazem.

22. Compound according to claim 9 or 10, in which the active group is derived from simvastatin or probucol.

23. Compound according to claim 9 or 10, in which the active group is derived from an aptamer or antisense oligonucleotide.

24. Therapeutic agents that contain a compound according to one of claims 9 to 23, dissolved, emulsified or suspended in an aqueous medium and the adjuvants, additives and/or stabilizers that are commonly used in galenicals.
